(19) United States
(12) Patent Application Publication
(10) Pub. No.: US 2007/0030947 A1
(43) Pub. Date: Feb. 8, 2007

(54) X-ray device with improved efficiency

(76) Inventor: Stefan Popescu, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 10/557,118

(22) PCT Filed: Jul. 6, 2004
(86) PCT No.: PCT/EP04/03672

§ 371 Date: Nov. 16, 2005

(30) Foreign Application Priority Data
May 16, 2003 (DE) 103 22 137.9

(51) Int. Cl. H05G 1/60; A61B 6/00; G01N 23/00; G21K 1/12

(52) U.S. Cl. 378/19

(57) ABSTRACT

An x-ray computed tomography apparatus has at least one x-ray source, one or more first x-ray detector elements disposed opposite the x-ray source, with an examination volume disposed therebetween, one or more x-ray deflection elements, and one or more second x-ray detector elements. The ray deflection elements respectively deflect x-rays emitted by said x-ray source in different spatial angular ranges onto the respective x-ray detector elements. The deflected x-rays proceed either through the first region of the examination volume as well, or through different regions of the examination volume. Faster scanning times and reduced artifacts in multi-layer computed tomography scanning are thereby achieved.

### BACKGROUND OF THE INVENTION

1. Field of the Invention

The present invention concerns an x-ray computed tomography (CT) apparatus, of the type having at least one x-ray source, one or more x-ray detector elements opposite the x-ray source, with an examination volume between the x-ray source and the x-ray detector elements, whereby x-ray radiation in an angular range emitted by the x-ray source is directed through a region of the examination volume onto the x-ray detector elements without deflection.

2. Description of the Prior Art

X-ray apparatuses of the above-described type play a significant role in the field of imaging medical technology. In this field, digital image acquisition techniques are increasingly used in which the x-ray radiation penetrating through a subject positioned in the examination volume is detected with spatial resolution in order to generate a corresponding, spatially-resolved x-ray image. With the technique of x-ray computed tomography it is possible to reconstruct and to show to the user nearly arbitrary slice images through the examination subject.

In addition to x-ray CT apparatuses, simple x-ray irradiation apparatuses are also known. DE 101 39 384 A1 thus describes an x-ray apparatus for generation of difference images. In a difference image method, two x-ray acquisitions of the examination region are obtained with different monochromatic x-ray radiation and are subtracted from one another. For this purpose, in the disclosed x-ray apparatus two different angular ranges of the x-ray emission of an x-ray tube are used in which the x-ray radiation is deflected by multilayer x-ray mirrors and directed onto the examination region. These multilayer x-ray mirrors are fashioned such that, due to Bragg reflection, they lead to a wavelength-sensitive reflection. Both mirrors in this x-ray apparatus are designed differently in order to generate different types of monochromatic x-ray radiation. The central x-ray emission is blocked by a beam stop.

U.S. 2002/0126796 A1 discloses a fluoroscopy apparatus for a 3D visualization. For this, two x-ray beams are generated by deflection elements from the x-ray emission of an x-ray source. The two x-ray beams are directed onto the examination region and the respective images are displayed separated from each other to produce a stereoscopic image impression.

A computed tomography scanner includes, among other things, an x-ray tube, x-ray detectors and a patient positioning table. The x-ray tube and the x-ray detectors are arranged on a gantry that rotates around the patient positioning table or around an examination axis running parallel thereto (the z-axis) during the measurement. The patient positioning table can be moved along the examination axis relative to the gantry. The x-ray tube generates an x-ray beam that expands in a fan shape in a slice plane perpendicular to the examination axis. In examinations in the slice plane, this x-ray beam penetrates a slice of a subject (for example a body slice of a patient who is on the patient positioning table) and strikes the x-ray detectors opposite the x-ray tube. The angle at which the x-ray beam penetrates the body slice of the patient, and, if applicable, the position of the patient positioning table relative to the gantry vary continuously during the image acquisition with the scanner.

The intensity of the x-rays of the x-ray beam striking the x-ray detectors after penetration through the patient is dependent on the attenuation of the of the x-rays by the patient. Dependent on the intensity of the acquired x-ray radiation, each detector element of the x-ray detector generates a voltage signal that corresponds to a measurement of the total transparency of the body for x-rays from the x-ray tube at the corresponding x-ray detector element. A set of voltage signals of the x-ray detectors that correspond to attenuation data and acquired for a specific position of the x-ray source relative to the patient is designated as a projection. A set of projections acquired at various positions of the gantry during the rotation of the gantry around the patient is designated as a scan. The computed tomography scanner acquires many projections at various positions of the x-ray source relative to the body of the patient in order to reconstruct an image that corresponds to a two-dimensional slice image of the body of the patient or a three-dimensional image. The prevalent method for reconstruction of a slice image from acquired attenuation data is known as the filtered back projection method.

In numerous applications of computed tomography, it is necessary to implement the x-ray acquisition with optimally short acquisition time. This primarily concerns acquisitions of body regions or organs with high biokinetics such as, for example, image acquisitions of the heart of a patient, which continuously moves periodically. In fact, for the generation of three-dimensional x-ray exposures of the heart the possibility exists to use electron beam CT systems (EBCT) but, these systems are extremely expensive and additionally provide a worse image quality than conventional multi-slice CT systems of the third generation. One approach for acquisition of 3D x-ray images of the heart is synchronization of the x-ray irradiation and data acquisition with an EKG signal. This technique is known as an EKG-controlled scan (EKG gating). With such a timing device, x-ray irradiation and data acquisition are activated only when the heart is electrically and mechanically in approximately the same phase. A problem with this technique is that the gantry rotation is not necessarily synchronized with the heartbeat frequency. This leads to some projection directions possibly not being acquired dependent on the relationship of the rotation speed and the heartbeat frequency, such that the later reconstruction from the acquired data is difficult. In clinical practice, for x-ray CT imaging of the heart the patient is therefore administered substances that artificially lower the heart rate. This, however, results in x-ray exposures of the heart in a modified state that can negatively affect the subsequent diagnostics.

One possibility for improvement of the above situation is to increase the number of projections that are acquired at every position of the gantry, for example by mounting a second x-ray source as well as a second x-ray detector on the gantry. A second x-ray source, however, increases the power consumption and the temperature generated within the gantry, as well as increasing the costs of the system as well as the maintenance, since the x-ray source is a component with a low lifespan.

A further problem of conventional x-ray apparatuses is the low energy efficiency of the x-ray tubes used therein. Only approximately 1% of the power consumed by these x-ray tubes is converted into x-ray energy, while 99% is emitted as heat. A further disadvantage of conventional x-ray tubes is that, dependent on the system, the x-ray emission surface of the anode that is struck by electron irradiation causes the x-ray radiation to be emitted in a very large spatial angular range, but only a small region thereof that proceeds through the exit window of the x-ray tube can be directly used for the generation of the x-ray images. This results from the requirement of the x-ray beam to be optimally narrow in one direction (known as the z-direction of the x-ray CT apparatus) but expanded in a fan shape in the direction perpendicular thereto, in order to be able to irradiate the corresponding slices of the examination subject in succession with the x-ray radiation beam. The remaining, unused portion of the x-ray emission is then absorbed within the x-ray tube or by a diaphragm.

For many applications, multi-slice CT apparatuses are used today that enable a better utilization of the generated x-ray emission as well as faster 3D x-ray image acquisitions. In these apparatuses, the x-ray is expanded in a cone shape in the z-direction and thus covers a larger subject volume per acquisition position of the gantry. A detector array composed of a number of parallel rows of x-ray detector elements is used in the side of the examination volume opposite the x-ray tube, such that a number of slices of the subject can be acquired in each acquisition position. This acquisition technique, however, entails new problems. Different effective focus sizes that depend on the position of the slice on the z-axis, and thus cause slice-dependent artifacts, result for each of the irradiated slices. Furthermore, given a beam significantly widened in a cone shape in the z-direction, artifacts are produced due to the conical expansion that can be corrected for a number of slices up to 16 via elaborate techniques but, given more than 16 slices they must be accepted. A further problem in the cited geometry is that only partial coverage of the examination subject can be achieved because conical regions remain (in particular at both ends in the z-direction) that are not penetrated by x-ray radiation. These regions likewise lead to artifacts in the reconstructed three-dimensional x-ray image.

To increase the spatial resolution and to cover a larger examination volume in the z-direction, multi-slice CT systems require a smaller focus size as well as a higher power of the x-ray tube. In addition to this, heart imaging with computed tomography requires a higher rotation speed of the gantry and a shorter scan time, such that a further increase of the tube power is necessary. Since the presently-used tube powers already trend in the direction of the 100 kW range, a pressing need exists to improve the efficiency of the energy utilization of the x-ray tubes in computed tomography apparatuses. This extends the lifespan of the x-ray tube and improves the availability of the CT apparatuses since long cooling pauses for the x-ray tube can be prevented.

U.S. Pat. No. 6,252,925 describes an x-ray CT apparatus with stationary x-ray source, in which the x-ray radiation is guided towards the examination volume by waveguides. Using a sequential coupling of the x-ray radiation into the fibers of the waveguide, for example with a rotating polygon mirror or a rotating aperture disc, a scan of the examination volume can be conducted without the use of a rotary frame.

An object of the present invention is to provide an x-ray apparatus, in particular an x-ray CT apparatus, that enables a shortening of the x-ray acquisition times without additional artifacts in the x-ray image and additionally exhibits an improved energy efficiency.

This object is achieved in accordance with the invention by an x-ray CT apparatus having an x-ray source, one or more first x-ray detector elements opposite the x-ray source with an examination volume between the x-ray source and the x-ray detector elements, with x-ray radiation from a first spatial angular range of the x-ray emission of the x-ray source being directed through a first region of the examination volume onto the first x-ray detector elements without deflection, and having one or more x-ray deflection elements as well as one or more further x-ray detector elements or groups of x-ray detector elements arranged on the x-ray apparatus such that x-ray radiation from one or more further spatial angular ranges of the x-ray emission of the x-ray source is directed through the first region or one or more of the further regions of the examination volume onto the further x-ray detector elements by the one or more ray deflection elements.

In the present x-ray apparatus, the x-ray emission emanating from the anode of known x-ray tubes ensues is utilized in a larger spatial angular range than for the generation of a single x-ray beam necessary for the irradiation of the subject. The x-ray radiation of these previously-unused spatial angular ranges is precisely directed onto the subject to be examined in the examination volume by the one or more additional ray deflection elements. Further x-ray detector elements are then correspondingly arranged on the opposite side in order to detect the irradiation of the subject with these further x-ray beams, i.e. to measure the attenuation of these x-rays caused by the body. The ray deflection elements are arranged such that they irradiate either the same region of the subject to be examined at a different viewing or projection direction or a further region, preferably offset in the z-direction, at the same projection direction. The number of the ray deflection elements and further x-ray detector elements depends on the intended effect and is only limited by the geometry and the spatial distribution of the x-ray emission of the x-ray source. A distinct improvement of the energetic efficiency of the x-ray apparatus is achieved by the better utilization of the x-ray emission of the x-ray source enabled with the ray deflection elements. In this manner, additional virtual x-ray sources are directly achieved by the ray deflection elements given use in x-ray CT apparatuses, with which unused x-ray quanta can be made usable for the x-ray acquisitions.

A further advantage of these one or more additional ray deflection elements with the associated x-ray detector elements is that additional projections are acquired at every position of the gantry—without having to increase the x-ray power—such that the acquisition speed can be increased in comparison with conventional x-ray CT apparatuses. The acquisition possibilities of moving body parts such as the heart are thereby directly, distinctly improved. In the present x-ray apparatus, depending on the design, alignment and arrangement of the ray deflection elements it is also possible to irradiate the subject with x-ray beams running parallel to one another, each with a substantially parallel ray cross-section, that, reduce the reconstruction effort in the reconstruction of the three-dimensional images from the measured raw data and enable an improved volume coverage in the z-direction without the artifacts that occur in known multi-slice apparatuses.

In the present x-ray apparatus, different elements known from the prior art can be used as ray deflection elements. An example is elements known as supermirrors that deflect the x-ray radiation using Bragg reflection. With such supermirrors, which are formed from a synthetically-generated multilayer system, the x-rays emitted from different spatial angular ranges can be arbitrarily deflected and shaped such that, for example, parallel or converging ray beams can be formed. The mirrors are parabolically shaped for this purpose and are formed by a number of crystalline layers in which the layer separation varies in a controlled manner in order to achieve the Bragg reflection. Such supermirrors are, for example, from “Parallel-Beam Coupling into Channel-Cut Monochromators Using Curved Graded Multilayers”, M. Schuster and H. Gobel, Siemens AG., J. Phys. D: Appl. Phys. 28 (1995) A270-A275; “Broad-band Focusing of Hard X-rays using a Supermirror”, Hoghoj, Joensen et al., OSA: Physics of X-ray Multilayer Structures (1994); “Measurement of multilayer reflectivities from 8 keV to 130 keV”, Hoghoj, Joensen et al., SPIE Vol. 2001, p. 354-359 (1994) ; “Gbbel Mirrors for Parallel-Beam Conditions”, Bruker AXS, Inc.—Analytical X-ray Systems, hftp://www.bruker-axs.com; “Twin Gobel Mirrors—The Real Parallel Beam Concept”, Bruker AXS, Inc.-Analytical X-ray Systems, http://www. bruker-axs.com. They enable the deflection and shaping of x-rays in the hard x-ray range and with high efficiency of 30-60%. In addition to these supermirrors, known crystals can also be used for the Bragg reflection, such that a parallel monochromatic x-ray can be generated via the combination of supermirror and such a typical Bragg reflector that serves as a monochromator.

A further possibility for design of the ray deflection elements for the present x-ray apparatus is the use of one or more bunches of hollow capillary tubes in which the x-ray radiation is conducted as in an optical fiber. Such bunches are normally comprised of hollow glass fibers and are also known as Kumakhov optics or polycapillary optics. They can be used for collimation, filtering and focusing both of x-ray radiation and of neutrons. Examples of such deflection elements are described in the publications “X-ray concentrator will expand window on high-energy universe”, Science@NASA, http://science.msfo.naoa.gov/newhome/headlines; “Capillary X-ray Optics-Introduction”, Center for X-ray Optics, University of Albany, N.Y., http://www.albany.edu/x-rayoptics/intro.html; “Parallel Beam X-Ray Diffraction, Application notes 201 and 202”, X-RAY Optical Systems Inc., Albany-N.Y., USA, http://www.xos.com. With such polycapillary optics it is possible, by appropriate curvature of the capillaries, to deflect the x-ray radiation arriving from a spatial angular range and to shape it in a nearly arbitrary manner. This enables the detection of a broad angular range and larger energy ranges (200 eV-30 keV) with a high efficiency of 10-50%. The technique is based on the total reflection of the x-ray radiation within the hollow glass capillaries, which exhibit a diameter between 5 and 50 μm. X-rays that enter into these capillaries at the critical angle are conducted along the capillary channels nearly without loss. With such optics it is also possible to focus the x-ray radiation, for example onto focus diameters of 20 μm or less, in order to be able to generate a higher x-ray flow with lower power of the x-ray tube. The use of such optics as ray deflection elements thus achieves particular advantages for x-ray CT apparatuses, since a larger spatial angular range of the x-ray radiation can be detected and the x-ray radiation can be shaped nearly arbitrarily, in particular for generation of a quasi-parallel ray beam. This technique additionally reduces the scattering and increases the transmission of primary x-ray quanta, such that a higher contrast is achieved at a reduced x-ray dose for the patient. The x-ray image can also be enlarged or shrunk due to the nearly arbitrary shaping capability.

In an embodiment of the present x-ray apparatus, the ray deflection elements are arranged such that the first region of the examination volume irradiated with the x-ray radiation from a first spatial angular range is also irradiated by x-ray radiation from the further spatial angular ranges, conducted onto the further x-ray detector elements via the ray deflection elements. The simultaneous irradiation of the subject from various projection directions is enabled without having to increase the x-ray power or having to provide further x-ray sources. Most notably, given use in an x-ray computer tomograph in which the x-ray source, the ray deflection elements and the x-ray detectors are arranged on the gantry, a faster scan can be conducted in this manner since a plurality of projection directions can be simultaneously acquired depending on the position of the gantry. Both ray deflection elements are thereby preferably arranged on both sides of the x-ray source in the same plane, perpendicular to the z-axis in which the x-ray source also lies, such that the three projection directions hereby generated lie in one plane.

The present x-ray apparatus is naturally not limited to two ray deflection elements. Rather, depending on the desired effect only one additional ray deflection element or distinctly more than two ray deflection elements can also be used together with the corresponding x-ray detector elements. An embodiment as a C-arm apparatus or as a simple x-ray apparatus without a rotating gantry is also possible.

In a further advantageous embodiment, the present x-ray apparatus is used as a multi-slice CT x-ray apparatus. The ray deflection elements as well as the groups of x-ray detector elements are thereby arranged on the x-ray apparatus such that regions of the examination volume lying in series in the axis direction of the rotation axis of the gantry (which corresponds to the z-axis) are irradiated by x-ray beams in a plurality of essentially parallel planes. The one or more x-ray beams of each of these planes thereby irradiate one or more slices of the examination subject that can then be correspondingly reconstructed. The x-ray radiation can thereby be shaped by the ray deflection elements such that they generate a parallel x-ray beam in the plane or a ray beam widened in a fan shape in the plane. Depending on the width of this x-ray beam in the direction perpendicular thereto (the z-direction), one or also more slices can be acquired via each of these x-ray beams. Given the acquisition of a plurality of slices, a corresponding plurality of rows of x-ray detector elements must naturally be provided on the opposite side of the examination volume. In known multi-slice x-ray CT apparatuses, these rows of x-ray detector elements are already realized in the form of a large-area detector array that can also be used in the same manner in the present x-ray apparatus. The number of the slices that can be acquired per ray deflection element thereby depends only on the ray expansion or the ray thickness (given parallel ray cross-section) in the z-direction, which can be predetermined by suitable design of the ray deflection elements.

Since the same volume can now be irradiated with approximately parallel x-ray beams, this embodiment of the present x-ray apparatus prevents the artifacts that are caused in the known apparatuses by the significant conical expansion of the x-ray beam in the x-direction. In the same manner, the only partial exposure that occurs at both ends of the examination region in the z-direction given conventional conical x-ray beams is prevented. In particular, given otherwise identical contrast a distinctly reduced x-ray power must be adjusted since the previously unused x-ray emission is used in the x-ray acquisition.

In a further embodiment of the present x-ray apparatus, a ray deflection element is used not only to cover the entire extent of the examination region perpendicular to the z-direction, but also to cover a number of adjacent ray deflection elements. In this embodiment, the examined region is therefore irradiated with a number of parallel x-ray beams in two dimensions. With a suitable design of the ray deflection elements, this enables the improved usage of scattered-ray grids on the detector side so that the signal-to-noise ratio of the exposures can be distinctly increased. Each individual ray deflection element thereby shapes the x-ray beam so that it penetrates the examination subject approximately in parallel or is focused on the respective opposite x-ray detector element over which a cell-like scattered-ray grid is arranged.

The design of the present x-ray apparatus also enables a particular shape of the electron beam focus on the rotating x-ray anode as in the known x-ray tubes of the prior art. Given a generation of this focus as a line in the radial direction relative to the rotation of the anode, this radial line focus is imaged by each ray deflection element as a line running in the z-direction on the x-ray detector elements. This design enables a better heat dissipation in the rotating anode via reduction of the peak power in the focal band, i.e. the focus track on the rotating anode surface. This leads to a higher (Hounds Filed unit) capacity of the x-ray tube.

The use of a larger number of ray deflection elements that are arranged both in series in the z-direction and next to one another enables (by suitable design of these ray deflection elements) the generation of a significantly larger electron focus on the x-ray anode than has previously been realizable. In the previous technique, an optimally small focus is generated in order to achieve an optimally punctiform x-ray source. Given a suitable design of the ray deflection elements of the present x-ray arrangement, different small regions of the focus of the anode can be mapped by these ray deflection elements, such that a each ray deflection element uses a different x-ray source. A number of nearly punctiform x-ray sources is achieved that are determined, not by the area of the focus of the anode, but rather solely by the power of the imaging optic. The focus of the x-ray anode thus can be designed very large, such that the local heat load of the anode is distinctly reduced.

### DESCRIPTION OF THE DRAWINGS

FIG. 1 schematically illustrates the basic components of a conventional x-ray computed tomography apparatus.

FIG. 2 schematically illustrates the x-ray emission from the x-ray source in the conventional apparatus of FIG. 1, as well as the portion of the x-radiation thereof that is conventionally utilized for imaging.

FIG. 3 schematically illustrates an embodiment of an x-ray computed tomography apparatus constructed and operating in accordance with the principles of the present invention.

FIG. 4 schematically illustrates a further embodiment of an x-ray computed tomography apparatus constructed and operating in accordance with the principles of the present invention.

FIG. 5 is a side view of the conventional x-ray emission, and utilized portion thereof, shown in FIG. 2.

FIG. 6 shows an example of the x-ray distribution in the z-direction of a conventional multi-slice x-ray computed tomography apparatus.

FIG. 7 schematically illustrates an example for x-ray guidance in an x-ray apparatus in accordance with the invention.

FIG. 8 shows another example for x-ray guidance in an x-ray apparatus in accordance with the present invention.

FIG. 9 shows a further example for x-ray guidance in an x-ray apparatus in accordance with the invention.

### DESCRIPTION OF THE PREFERRED EMBODIMENTS

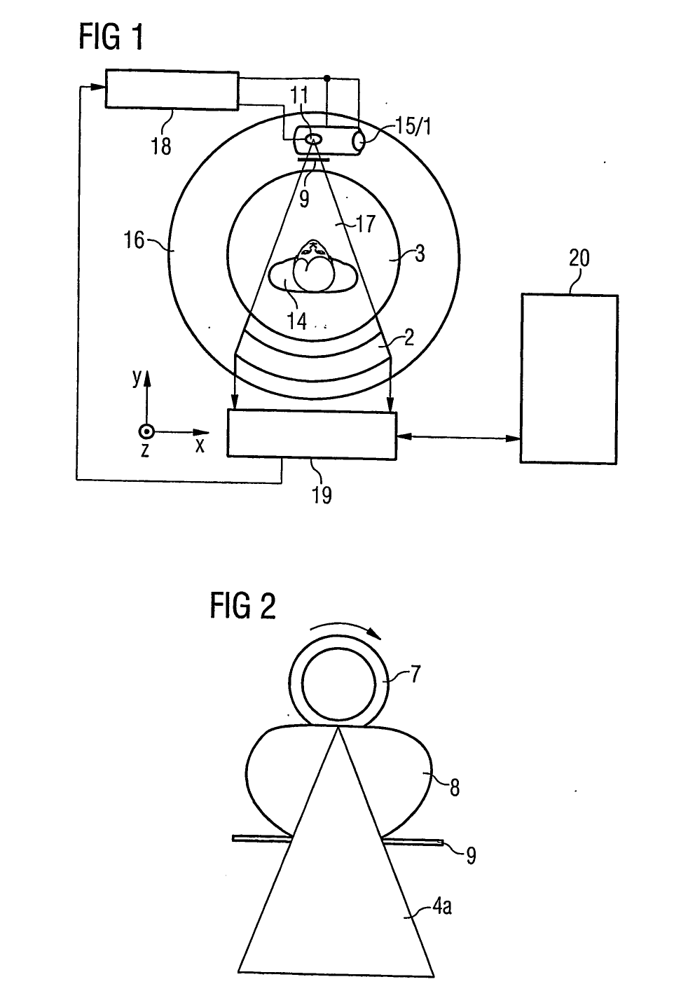

FIG. 1 schematically shows a part of the design of an x-ray CT apparatus that forms the basis of many embodiments of the present x-ray apparatus, with the exception of the ray guidance of the x-ray radiation. The x-ray CT apparatus has an x-ray source in the form of an x-ray tube 15 that emits a fan-shaped x-ray beam 17 in the direction of a detector row with x-ray detector elements 2. Both the x-ray tube 15 and the detector elements 2 are arranged on a gantry 16 which can rotate continuously around a patient 14. The patient 14 lies on a patient positioning table (not shown in FIG. 1) that extends into the gantry 16. The gantry 16 rotates in an x-y plane of a Cartesian coordinate system x-y-z indicated in FIG. 1. The patient positioning table is movable along the z-axis, which corresponds to the slice thickness direction of the respective slices of the patient 14 to be shown. The expansion of the x-ray beam 17 in the z-direction (in the present representation the direction perpendicular to the drawing plane) is predetermined by the expansion of the focus 11 on the rotating anode of the x-ray tube 15 as well as by the diaphragm 9 arranged on the tube side, the diaphragm aperture of which diaphragm 9 is adjustable in the z-direction.

The x-ray tube 15 is supplied by a high-voltage generator 18 with a high voltage of, for example, 120 kV. A controller 19 serves for the activation of the individual components of the computed tomography apparatus (in particular the high-voltage generator 18, the gantry 16, the detector elements 2 as well as the patient bed (not shown)) for implementation of the measurement data acquisition. The measurement data are relayed to an image computer 20 in which the image reconstruction from the measurement data is conducted.

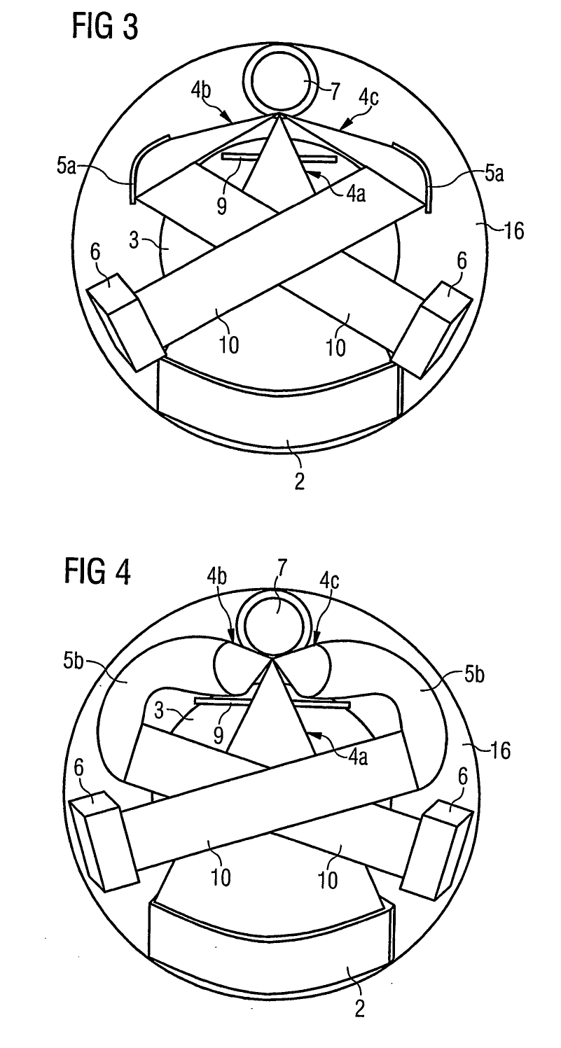

FIG. 2 schematically shows the distribution of the x-ray emission of an x-ray tube as it us used in x-ray apparatuses. In this representation, which shows a slice plane perpendicular to the z-axis (i.e. perpendicular to the rotation axis of the gantry of a CT apparatus), the disc-shaped anode 7 of the x-ray tube that rotates around its central disc axis during the generation of x-ray radiation is visible. Electron beams are generated in the x-ray tube and focused on a boundary region of the anode 7. X-ray radiation is released in a known manner from the x-ray emission surface formed by the focus by the impact of the accelerated electrons on the anode 7. The rotation of the anode 7 is necessary in order to prevent a too-severe local overheating leading to a destruction of the anode 7. The spatial distribution of the x-ray emission 8 emanating from the focus of the anode 7 is indicated in FIG. 2 in the shown plane. The x-ray emission 8 ensues nearly in the entire hemisphere. For x-ray acquisitions with the x-ray CT system, only a first spatial angular range 4a from this spatial distribution is used in order to obtain in the shown slice plane, a fan-shaped, expanded ray beam that emanates from the focus. For this purpose, a suitable diaphragm 9 is used that limits the first spatial angular range 4a. From FIG. 2 it is clear that only a small part of the x-ray quanta emitted by the anode 7 is used for the x-ray acquisition.

In the present x-ray apparatus, at least one part of this previously-unused x-ray emission 8 is likewise used for the generation of the x-ray exposure. For this, one or more ray deflection elements 5a, 5b are used that deflect further spatial angular ranges of the x-ray emission 8 through a region of the examination volume onto further x-ray detector elements 6. FIG. 3 shows an example for such an embodiment of the present x-ray apparatus. In this embodiment, which shows a section through the x-ray apparatus in the same plane as FIG. 2, supermirrors 5a are arranged on both sides of the diaphragm 9. The supermirrors 5a additionally deflect x-ray radiation emanating from the anode 7 at second 4b and third angular ranges 4c into the examination volume 3 on the subject to be examined. Further x-ray detector elements 6 arranged on the gantry 16 detect with spatial resolution the attenuation of the x-ray radiation caused by the subject. In this example, the supermirrors 5a are parabolically shaped such that they shape parallel x-ray beams 10 from the respective second 4b and third spatial angular ranges 4c. In this example, in addition to the main projection direction established by the diaphragm 9 two additional projection directions are therefore acquired at every position of the gantry 16. All x-ray beams penetrate the same region of the examination subject in the same slice plane. A faster scan can be realized in this manner without having to provide additional x-ray power. Such an embodiment is therefore in particular suitable for x-ray acquisitions of moving subjects of the body, for example the heart. The additional x-ray detector elements 7 must naturally be mounted at the suitable point of the gantry in order to detect the parallel x-ray beams 10.

FIG. 4 shows a further example for the present x-ray apparatus, in which [[a]] polycapillary optics 5b is used instead of the supermirror 5a. The further embodiment of this x-ray apparatus corresponds to the design of FIG. 3, such that at this point it is not discussed in detail again. The use of the polycapillary optics 5b instead of the supermirror 5a has the advantage that a larger spatial angular range can be converted into parallel x-ray beams 10 with the polycapillary optics 5b. The capillaries of the polycapillary optics 56 are suitably curved for the deflection of the x-ray radiation.

Additional virtual x-ray sources that enable an increased data acquisition speed without precipitating additional artifacts are thus generated with both exemplary embodiments of FIGS. 3 and 4. In known x-ray apparatuses, the increased data acquisition speed can only be achieved only with an increased rotation speed of the gantry. Furthermore, the use of the further spatial angular ranges of the x-ray emission of the x-ray source enables an increase of the energy efficiency of the apparatus. Due to the better utilization of the x-ray emission, it is now also possible to use additional monochromators (in the form of Bragg reflectors) in order to direct monochromatic or quasi-monochromatic x-ray radiation onto the subject. This was previously barely possible due to the low efficiency of the utilization of the x-ray radiation; however, in the examination of soft tissue it leads to the generation of a better image contrast at a reduced x-ray dose for the patient.

In the last-cited examples, the ray deflection elements were used in order to generate x-ray beams in the same slice as the primary ray beam. Naturally, dependent on the purpose of the application it is also possible with these further x-ray beams to expose a plurality of different slices. It depends merely on the arrangement and alignment of the ray deflection elements as well as the arrangement of the further detector elements. For example, it is possible to realize multi-slice x-ray CT apparatuses in which a larger volume region in the z-direction is covered by the additional ray deflection elements. Given the use of one or more ray deflection elements that shape parallel x-ray beams, the conical hollow regions of the examined volume remaining at both ends in the z-direction can also be acquired in this manner. This improves the volume coverage and the total dose efficiency.

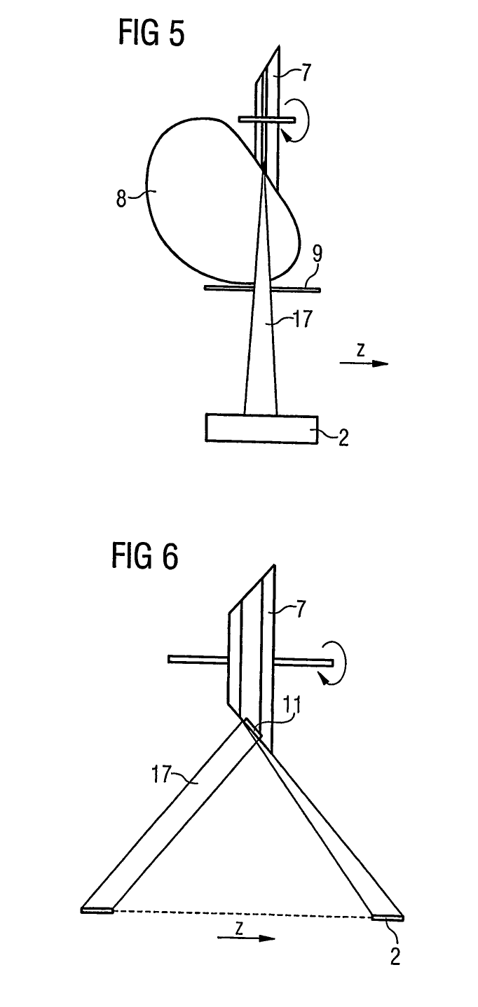

The following figures show examples for such embodiments of the present x-ray apparatus as a multi-slice x-ray CT apparatus. FIGS. 5 and 6, schematically show the previous relationships in apparatuses of the prior art. FIG. 5 schematically shows the anode 7 of the x-ray tube that rotates around its central disc axis. The representation shows a slice plane perpendicular to that of FIG. 2, i.e. a slice plane in which the z-axis or the rotation axis of the gantry also lies. The focal band, which forms the electron radiation and the rotation of the anode 7 via the focusing, is recognizable on the anode 7. FIG. 6 shows the large angular range in this shown plane in which the x-ray emission 8 ensues. Here as well the diaphragm 9 is again recognizable, which diaphragm significantly limits the spatial angular range in the z-direction in order to acquire optimally thin slices of the examined subject with the x-ray radiation, as this is the case in single-slice x-ray CT apparatuses.

In multi-slice CT x-ray apparatuses, the x-ray emission in the z-direction is not so significantly limited, as this is clear from FIG. 6. Here a conical x-ray beam 17 is also generated in the z-direction with a large aperture angle, which conical x-ray beam 17 strikes multiple rows of x-ray detector elements 2. Each slice results from the linear relationships between the delimitation of the focus 11 of the anode 7 and the delimitation of the respective row of x-ray detector elements 2. A number of slices can therefore be simultaneously acquired with this technique. However, as can be seen from FIG. 6, every row of x-ray detector elements 2 sees a different size of the focus 11, such that the dimension of the respective irradiated slice also varies in the z-direction. This leads to slice-dependent artifacts that can only be calculationally corrected given a lower number of simultaneously-acquired slices.

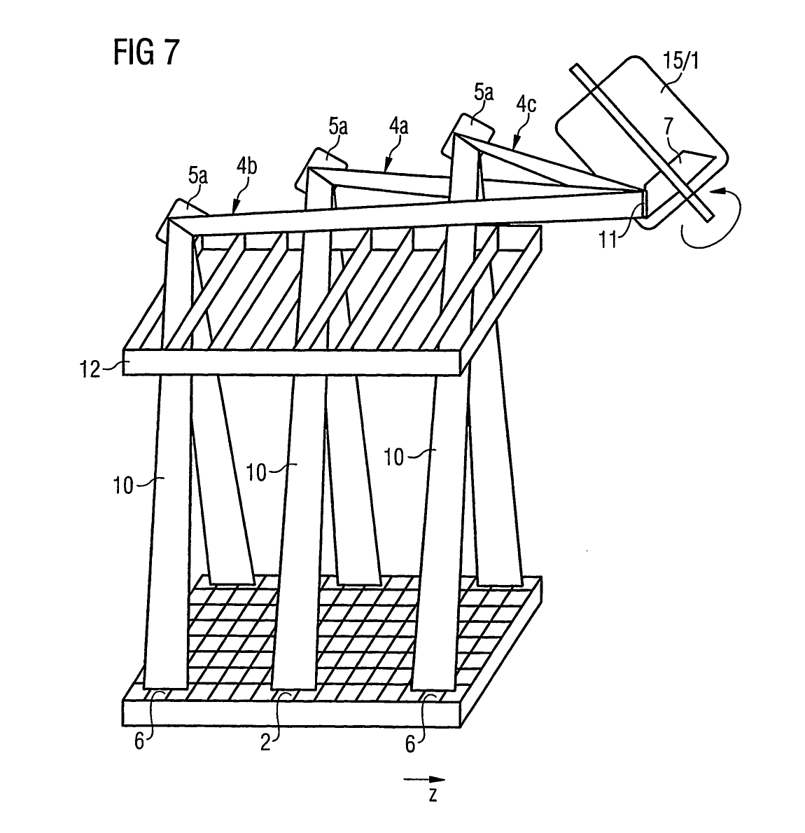

FIG. 7 now shows an embodiment of the present x-ray apparatus in which such artifacts are prevented. In this embodiment, parallel x-ray beams 10 arranged in series in the z-direction are generated with a number of ray deflection elements (of which, for clarity, only 3 are shown in FIG. 7), which x-ray beams 10 are expanded in a fan shape in the respective plane. In this example, all x-ray beams penetrating the subject are directed onto the subject via deflection elements, such that x-rays emanating directly from the x-ray source are no longer used. However, this is not necessarily the case. Different spatial angular ranges 4a, 4b, 4c of the x-ray emission of the anode 7 are used in turn with the supermirrors 5a used as ray deflection elements in the present case. This leads to the same advantages as they were already explained in connection with the embodiments of FIGS. 3 and 4. The x-ray detector elements 2, 6 used in this example exist in the form of a detector array that can be designed identical to the detector array of a conventional multi-slice x-ray CT apparatus. The individual rows (lying in series in the z-direction) of these x-ray detector elements 2, 6 define the respective slices. The x-ray beams 10 shaped with the ray deflection elements 5a can hereby run in parallel in the z-direction and charge one or more rows of x-ray detector elements 2, 6 with x-ray radiation. Multiple rows of x-ray detector elements 2, 6 are preferably covered with each of these x-ray beams 10. If N represents the number of the supermirrors 5a arranged offset in the z-direction, a number M of slices should be irradiated by these, whereby M≧N. A slightly conical expansion of the x-ray beams 10 in the z-direction is also possible without generating the known artifacts.

In this embodiment, it is also recognizable that a line-shaped focus 11 can be generated on the anode 7 in order to achieve an improved heat distribution on the rotating anode 7. The peak power in the focal band is thereby reduced. The line-shaped focus 11 that runs in the radial direction on the anode surface is hereby mapped as a line-shaped focus in the z-direction on the x-ray detector elements 2, 6, such that no loss of spatial resolution results from this.

The x-ray mirrors 5a can also be designed so that they focus the generated x-ray beams 10 onto a virtual focus behind the x-ray detector elements 2, 6 in order to increase the spatial resolution given the same focus size on the anode 7 and the power of the x-ray tube and to reduce extra-focal radiation. Extra-focal radiation degrades the modulation transfer function MTF and produces “halo artifacts” in head images, in particular in children. There are in fact compensation algorithms that, however, increase the noise in the images. Furthermore, it is known to use two-dimensional comb filters as scattered-ray grids on the detector in order to reduce the effects of extra-focal radiation. However, this has as yet not led to a complete reduction of the effects of extra-focal radiation since the patient himself is always still charged with such radiation, and moreover Compton photons can produce further artifacts.

The present embodiment of the x-ray apparatus enables the use of a one-dimensional, comb-shaped collimator in the z-direction that likewise eliminates possible inaccuracies in the surface of the x-ray mirror like the effect of extra-focal radiation. FIG. 7 shows such a comb-shaped collimator 12 on the side of the x-ray source.

A further advantage in the use of supermirrors 5a that deflect the x-ray radiation onto the examination. subject results from the particular design of these mirrors. Since approximately parallel x-ray radiation can be shaped with such supermirrors from a broader spatial rotating anode of the x-ray emission, additional Bragg reflectors can advantageously be used as monochromators in which x-ray radiation of a specific energy range is only reflected at a very narrow angular range of incidence. The parallel x-ray radiation generated by the supermirrors thereby prevents high losses. With the additional Bragg reflectors it is possible to separate the K_{α}- or the K₆₂-radiation from the remaining portion of the boundary radiation spectrum of the x-ray emission. Monochromatic or quasi-monochromatic x-ray beams can therefore be generated in this embodiment. This improves the signal-to-noise ratio in the x-ray exposures and leads to an improved dose contrast as well as to a reduction of the patient dose by gating out the high-energy portion from the x-ray spectrum.

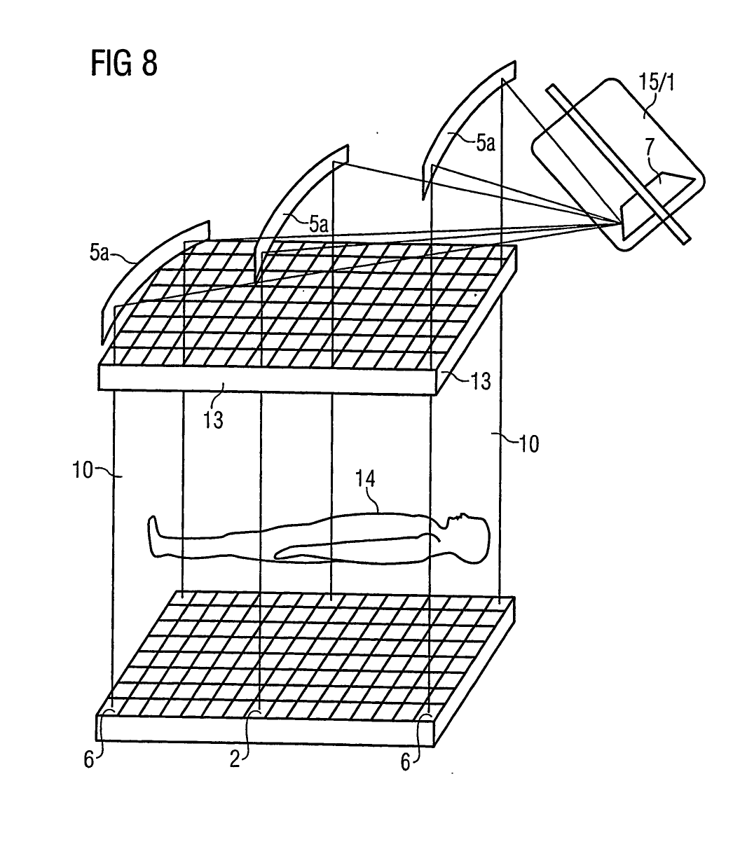

FIG. 8 shows a further exemplary embodiment of the present x-ray apparatus as a multi-slice x-ray CT apparatus. In this example, which is comparable to that of FIG. 7, the supermirrors 5a generate approximately parallel x-ray beams 10 over the entire slice region in each slice plane. These x-ray beams 10 are also preferably approximately parallel in the z-direction. The mirrors are distinctly widened in the plane perpendicular to the z-direction in comparison to the mirrors of FIG. 7, such that they include a distinctly larger angular range and thus also distinctly increase the number of the x-ray quanta available for the x-ray acquisition.

In such an embodiment, a cell-like collimator 13 can be used on the side of the x-ray source, which collimator 13 eliminates the effect of surface imprecisions on the mirror surfaces and ensures the generation of two-dimensional, parallel x-ray beams 10 in the direction of the x-ray detector elements 2, 6. Extra-focal radiation as well as ring artifacts are also at least reduced via use of such a collimator 13.

A further advantage of such an embodiment with two-dimensional, parallel x-ray beams is that the calculation effort for the image reconstruction is distinctly reduced in comparison with the use of fan-shaped x-ray beams. This reduces the reconstruction time, since in particular the reconstruction steps of the correction of cone-ray artifacts as well as the projection resorting are avoided.

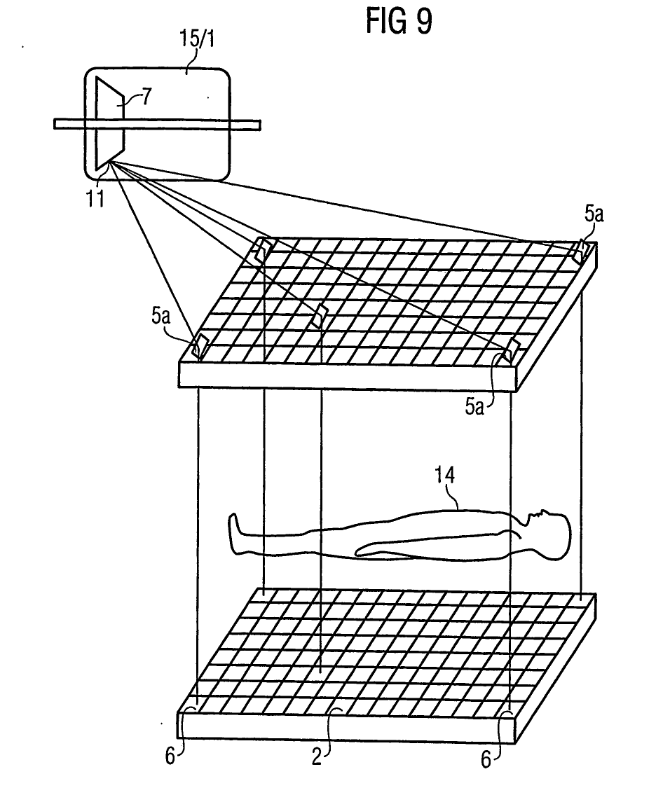

Finally, FIG. 9 shows a further exemplary embodiment that is very similar to that of FIG. 8. In this example, an array of mirrors 5a is used so that a number of parallel x-ray beams next to one another are generated in every slice plane. In this embodiment, each individual ray deflection element 5a is designed so that it maps a different small region of the x-ray emission surface of the anode 7 onto the respective x-ray detector elements 2 or 6. It is thereby possible to generate a very large focus 11 on the anode 7 without reducing the resolution of the x-ray exposure. With such an arbitrarily large focus, that is limited only by the size of the anode, the power of the x-ray source can be increased without immediately precipitating a local overheating.

The last-cited embodiments therefore enable a large and complete coverage of an examination volume in the z-direction given only a single rotation around the patient. This significantly reduces the scan time and thus the throughput of the CT apparatus. Furthermore, power losses due to heat generation are significantly reduced. The faster scan time with larger volume coverage enables the acquisition of body regions or organs with high biokinetics without significant movement artifacts. The embodiments additionally eliminate slice-dependent focus sizes and artifacts resulting from them. By the use of microactuators for the movement of the deflection elements, it is additionally possible to modulate the size of the parallel ray beams in the z-direction and to limit the surface exposed to the x-ray radiation in this manner. Furthermore, the mirrors can be optimally adapted to the respective x-ray tube via such microactuators.

Although modifications and changes may be suggested by those skilled in the art, it is the invention of the inventors to embody within the patent warranted heron all changes and modifications as reasonably and properly come within the scope of their contribution to the art.

I claim as my invention:

1.-16. (canceled)

17. An x-ray computed tomography apparatus comprising:
an x-ray source that emits x-ray radiation;
at least one first x-ray detector element disposed opposite said x-ray source, with an examination volume therebetween, x-ray radiation from said x-ray source in a first spatial angular range proceeding through a first region of said examination volume onto said at least one first x-ray detector element;
at least one second x-ray detector element; and
at least one ray deflection element disposed in said x-ray radiation to deflect x-radiation in at least one further spatial angular range through a region of said examination volume, selected from the group consisting of said first region and at least one second region, onto said at least one second x-ray detector element.

18. An apparatus as claimed in claim 17 comprising a plurality of second x-ray detector elements and a like plurality of ray deflection elements, said plurality of ray deflection elements deflecting x-ray radiation emitted by said x-ray source in respectively different spatial angular ranges respectively onto said plurality of second x-ray detector elements.

19. An apparatus as claimed in claim 18 wherein said plurality of second x-ray detector elements are arranged in a plurality of groups, with all second x-ray detector elements in each group being struck by x-ray radiation deflected by a single one of said plurality of ray deflection elements.

20. An apparatus as claimed in claim 17 wherein said ray deflection element deflects said x-ray radiation in said further spatial angular range through said first region of said examination volume, so that said first region of said examination volume is irradiated from two different projection directions.

21. An apparatus as claimed in claim 17 comprising a gantry surrounding said examination volume, on which said x-ray source, said at least one first x-ray detector element, said at least one second x-ray detector element, and said at least one ray deflection element are mounted, said gantry being rotatable around a rotation axis proceeding through said examination volume.

22. An apparatus as claimed in claim 20 wherein said at least one ray deflection element and said at least one second x-ray detector are mounted on said gantry so that said x-ray radiation emitted from said x-ray source in said further spatial angular range proceeds through a further region of said examination volume, said first region and said further region being respectively disposed in substantially parallel planes through which said axis proceeds.

23. An apparatus as claimed in claim 21 wherein said at least one ray deflection element shapes said x-ray radiation in said further spatial angular range into an x-ray beam that expands in a fan-shape in one of said parallel planes.

24. An apparatus as claimed in claim 20 comprising a plurality of second x-ray detector elements respectively disposed in detector rows, and wherein said at least one ray deflection element deflects said x-ray radiation in said further spatial angular range onto a plurality of said detector rows.

25. An apparatus as claimed in claim 21 comprising a plurality of second x-ray detectors disposed in a two-dimensional array, and comprising two ray deflection elements respectively deflecting x-ray radiation emitted by said x-ray source in different further spatial angular ranges to proceed in respective parallel planes through further regions of said examination volume onto said array.

26. An apparatus as claimed in claim 17 wherein said x-ray source comprises a rotating anode and an electron source that emits an electron beam that strikes a portion of a surface of said rotating anode to emit said x-ray radiation.

27. An apparatus as claimed in claim 25 wherein said portion of said surface of said rotating anode is a line proceeding radially on said rotating anode.

28. An apparatus as claimed in claim 25 comprising a plurality of ray deflection elements and a plurality of second x-ray detector elements, said plurality of ray deflection elements respectively deflecting different further spatial angular ranges of said x-ray radiation onto said plurality of second x-ray detector elements, to map respectively different regions of said surface of said rotating anode respectively onto said plurality of said second x-ray detector elements.

29. An x-ray apparatus as claimed in claim 17 wherein said at least one ray deflection element is a parabolically-shaped supermirror formed by a crystalline multi-layer structure.

30. An apparatus as claimed in claim 28 comprising a monochromator, formed by a Bragg reflector, disposed between said at least one ray deflection element and said examination volume.

31. An apparatus as claimed in claim 28 comprising a plurality of ray deflection elements respectively deflecting x-ray radiation in a plurality of further spatial angular ranges into a plurality of parallel x-ray beams.

32. An apparatus as claimed in claim 28 comprising a plurality of ray deflection elements respectively deflecting x-ray radiation in a plurality of further spatial angular ranges into a plurality of converging x-ray beams.

33. An apparatus as claimed in claim 17 wherein said at least one ray deflection element is formed by polycapillary optics.

34. An apparatus as claimed in claim 32 comprising a monochromator, formed by a Bragg reflector, disposed between said at least one ray deflection element and said examination volume.

35. An apparatus as claimed in claim 32 comprising a plurality of ray deflection elements respectively deflecting x-ray radiation in a plurality of further spatial angular ranges into a plurality of parallel x-ray beams.

36. An apparatus as claimed in claim 32 comprising a plurality of ray deflection elements respectively deflecting x-ray radiation in a plurality of further spatial angular ranges into a plurality of converging x-ray beams.
